(19) United States
(12) Patent Application Publication
(10) Pub. No.: US 2020/0296995 A1
(43) Pub. Date: Sep. 24, 2020

(54) MILK REPLACERS THAT INCLUDE TEXTURED SOY PROTEIN AND METHODS OF FEEDING THE SAME

(71) Applicant: PURINA ANIMAL NUTRITION LLC, Shoreview, MN (US)

(72) Inventors: Robert C. Musser, Woodbury, MN (US); Thomas Earleywine, Cottage Grove, WI (US)

(21) Appl. No.: 16/358,159

(22) Filed: Mar. 19, 2019

(51) Int. Cl. A23K 20/147; A23K 10/30; A23K 50/60; A23K 50/10; A61K 38/16

(52) U.S. Cl. CPC A23K 20/147; A23K 10/30; A61K 38/168; A23K 50/10; A23K 50/60

(57) ABSTRACT

Milk replacers include milk and non-milk proteins, and at least a portion of the non-milk protein is provided by textured soy protein. The textured soy protein is ground and extruded such that it is substantially free of anti-nutritional factors that are detrimental to young animal health or development. The milk replacers with textured soy proteins are fed to young animals, who demonstrate equal or improved performance compared to young animals fed milk replacers with the same amount of total protein and same amount of soy protein, but the soy protein is from other than textured soy protein.

### TECHNICAL FIELD

The present disclosure relates generally to methods of feeding milk replacers to young animals, and more particularly to feeding young animals milk replacers including protein, at least a portion of which is a textured soy protein.

### BACKGROUND

Livestock animals are a commodity and are raised to produce milk and meat. The time it takes livestock to mature, particularly to gain weight, is important when assessing whether the animal is ready to produce milk or is ready for market. A number of feeding systems have been used to enhance weight gain of livestock beginning at a young age, and may include feeding techniques prior to and after weaning. Such techniques may involve providing milk replacers to the animals that generally mimic the milk produced from the post-partum parent animal in terms of protein, fat and carbohydrate content. The milk replacer may be supplemented with vitamins, minerals, medication and other compositions that may benefit the young animals. This may, for example, reduce the age of freshening or the onset of lactation of a dairy cow, thereby reducing the cost of milk production. Increased weight gain of livestock from an early age may also reduce the cost of beef production.

A concern for producers is whether livestock animals are receiving adequate nutrients. When the livestock animals refuse feed, intake is decreased, which may be problematic from both the standpoint of the health of the animal and to the cost of milk or meat production. Differences in feed intake by animals impact rate of weight gain and ultimately body size. Young animals are more difficult to adequately manage due to their specific housing, dietary and husbandry needs.

Although various feeding systems have been practiced to enhance weight gain, these feeding systems have not fully optimized methods for increasing the rate and uniformity of weight gain by the livestock animals from a young age. Accordingly, producers are in need of new approaches to feeding young livestock animals as well as other young animals that enhance weight gain rates, increase feed efficiency, and/or are cost-effective.

### SUMMARY

Milk replacers and methods of feeding milk replacers to young animals are disclosed herein. According to one implementation, a milk replacer includes at least about 20 percent protein by dry weight. At least about 5 percentage units of the protein by dry weight is provided by a ground, extruded textured soy protein substantially free of anti-nutritional factors.

In additional or alternative embodiments, at least about 65 percent of the textured soy protein may pass through a 37 μm mesh. About 5 percent to about 70 percent of the protein by dry weight may be provided by the textured soy protein. About 30 percent to about 95 percent of the protein by dry weight may be provided by at least one milk protein. The textured soy protein may include about 20 percent to about 60 percent by weight protein. The textured soy protein may be present at about 5 percent to about 40 percent by dry weight of the milk replacer. At least a portion of protein of the textured soy protein may be at least partially denatured. The textured soy protein may be suspended in the milk replacer when the milk replacer is hydrated.

According to another implementation, a method of feeding a young animal includes providing a milk replacer to the young animal, the milk replacer including at least about 20 percent protein by dry weight. At least about 5 percentage units of the protein by dry weight is provided by a ground, extruded textured soy protein substantially free of anti-nutritional factors.

In additional or alternative embodiments, at least about 65 percent of the textured soy protein may pass through a 37 μm mesh. The young animal may experience improved performance in response to ingesting the milk replacer. The improved performance may include an increased feed-to-gain ratio and may occur by six weeks after birth or earlier. The young animal may be a calf.

According to another implementation, a method of feeding a young animal includes providing a textured soy protein in a milk replacer, the textured soy protein present at about 4 percent to about 40 percent by dry weight of the milk replacer and the textured soy protein having been extruded and ground; and providing the milk replacer to the young animal.

In additional or alternative embodiments, at least about 65 percent of the textured soy protein may pass through a 37 μm mesh. The textured soy protein may include about 20 percent to about 60 percent by weight protein. The milk replacer powder may include about 20 percent to about 30 percent protein by dry weight. The young animal may experience improved performance in response to ingesting the milk replacer. The improved performance may include an increased feed-to-gain ratio.

### DETAILED DESCRIPTION

### Overview

Young animals separated from their mother at birth are often fed milk replacers for nourishment until the animals are weaned. Milk replacers may include a blend of protein and fat in an amount that mimics milk produced by the female of the species. They are typically produced as dry powders and are hydrated in water immediately prior to feeding. Milk replacers disclosed herein and methods of feeding the same are applicable to young animals such as calves, lambs, kids, other young ruminants, piglets, other young livestock animals, foals, young zoo animals and young companion animals. The young animals may be fed individually or in a group setting. The milk replacers disclosed herein include textured soy proteins and may be conventional or full potential milk replacers. Disclosed conventional milk replacers include at least about 20 percent protein by dry weight, which is provided by a combination of non-milk proteins (including textured soy proteins) and milk proteins. Conventional milk replacers are often fed at a rate of at least about 1 pound per head per day on a dry weight basis. Disclosed high protein milk replacers, also referred to as full potential milk replacers, include at least about 25 percent protein by dry weight, which is provided by a combination of non-milk proteins (including textured soy proteins) and milk proteins. Full potential milk replacers are often fed in an enhanced setting, such as at least about 1.8 pounds per head per day on a dry weight basis.

Non-milk proteins have been used in animal milk replacers for decades as a means to reduce cost of products. Both animal and plant alternative proteins, referred to as non-milk proteins, have been used in this application.

One example of a non-milk protein used in milk replacers is soy protein. An early and continued source of soy protein has been unprocessed defatted soy flour. Defatted soy flour is generally ground such that the majority of it passes through a 200-mesh filter. However, soy flour is largely insoluble in the water used to hydrated milk replacers, so the soy flour can separate and settle out, which can lead to the provision of inconsistent nutrition in the field. Also, the inclusion of unprocessed defatted soy flour is known to result in poor calf performance and increased calf morbidity and mortality, likely because anti-nutritional factors in the soy flour have not been destroyed.

Hydrolyzed soy protein modified is another source of soy-derived non-milk protein that has been included in milk replacers. Hydrolyzed soy protein modified is produced using soy white flakes processed with alcohol and a reducing agent to cause hydrolysis of a portion of the soy protein into its constituent amino acids. Processing techniques result in a reduced antigenicity of hydrolyzed soy protein modified, which results in this protein source being more digestible in animals compared to other soy-based protein sources such as soy flour. U.S. Reissue Patent RE43929, entitled “Method of Processing Soy Protein,” and U.S. Pat. No. 4,450,176 entitled “Method of Treating Soybean Material,” describe methods of processing soy protein to produce hydrolyzed soy protein modified, and are incorporated by reference in their entirety for any useful purpose. The production method for hydrolyzed soy protein modified can be time-consuming. The method can include the use of significant amounts of 95 percent ethanol and/or the significant production of spent alcohol byproducts, which can be difficult or expensive to dispose.

Milk Replacers Containing Textured Soy Protein

Milk replacers disclosed herein include textured soy protein. Textured soy protein is a processed food product derived from soy flour, soy meal, soy white flakes, or soy protein concentrate.

The soy starting material may be extruded such that at least a portion of the proteins therein are at least partially denatured. The extrusion may be performed at a high enough temperature and/or a high enough pressure to plasticize the proteins. In one example, soy starting material is extruded at about 325 F for about 25 seconds to about 30 seconds. The textured soy protein may be free or substantially free of active anti-nutritional factors. The textured soy protein may be a rubbery, expanded material compared to the soy starting material. The textured soy protein may be prepared in the absence of a chemical reaction, such as without the addition of reagents to denature the proteins.

After extrusion, the textured soy protein may be dried to, for example, <20 percent moisture, <15 percent moisture, <12 percent moisture, <10 percent moisture, or <8 percent moisture.

The textured soy protein may have a protein content of from about 20 percent to about 60 percent, about 20 percent to about 55 percent, about 20 percent to about 50 percent, about 20 percent to about 45 percent, about 20 percent to about 40 percent, about 20 percent to about 35 percent, about 20 percent to about 30 percent, about 25 percent to about 60 percent, about 30 percent to about 60 percent, about 35 percent to about 60 percent, about 40 percent to about 60 percent, or about 45 percent to about 60 percent. In one example, the textured soy protein has a protein content of about 25 percent. In another example, the textured soy protein has a protein content of about 50 percent to about 51 percent.

The textured soy protein disclosed herein may fit the definition of AAFCO 84.64: “Textured Soy Protein Product is made from defatted soy flour mixed with water and/or steam, extruded and then dried.” The textured soy protein disclosed herein may fit the definition in the Handbook of Food Proteins (M. N. Riaz, 2011, Chap. 15): “fabricated palatable food ingredients processed from an edible protein source including among others soy grits, soy protein isolates, and soy protein concentrates with or without suitable option ingredients added for nutritional or technological purposes.”

After extrusion and/or drying, the textured soy protein may be ground to any size suitable for inclusion in a hydrated milk replacer. The textured soy protein is not soluble in hydrated milk replacer and the grinding may help the textured soy protein be suspended or stay in suspension when added to a milk replacer. Grinding may help prevent the textured soy protein from separating and sinking to the bottom of a vessel that holds a milk replacer. Maintaining the textured soy protein in suspension in a milk replacer can aid in consistent distribution of, and nutrition provided by, the textured soy protein when the milk replacer is allocated to young animals.

The textured soy protein may be ground such that up to about 100 percent, at least about 98 percent, at least about 95 percent, at least about 90 percent, at least about 85 percent, at least about 80 percent, at least about 75 percent, at least about 70 percent, or at least about 65 percent of the textured soy protein passes through one or more of the following filter sizes: 140 mesh (105 μm), 170 mesh (88 μm), 200 mesh (74 μm), 230 mesh (63 μm), 270 mesh (53 μm), 325 mesh (44 μm), and 400 mesh (37 μm). In one example, at least about 80 percent to about 85 percent passes through a 325 mesh (44 μm) filter. In another example, at least about 80 percent to about 85 percent passes through a 400 mesh (37 μm) filter. In another example, at least about 98 percent passes through a 400 mesh (37 μm) filter.

The textured soy protein may be ground approximately as fine as hydrolyzed soy protein modified, which may pass through a 400 mesh (37 μm) filter. The textured soy protein may be ground finer than soy flour, which may pass through a 200 mesh (74 μm) filter.

By comparison, other sources of soy protein may be spray dried instead of ground or may be ground but not as fine as the disclosed textured soy protein. In one example, a soy isolate is spray dried and not ground. In another example, a soy protein concentrate is ground only enough to pass through an 80 mesh (177 μm) filter to 100 mesh (149 μm) filter.

The extruded, ground textured soy protein may be free of or substantially free of one or more active agents present in soy that are known to negatively effect young animals. Examples of these agents include anti-nutritional factors, trypsin inhibitors, and allergens, such as glycinin and beta conglycinin. The agents may not be present or may be present but not active. One or more of the extrusion, drying, and fine grinding may destroy or disable the agents such that they no longer have a detrimental effect on young animals.

The milk replacers of the present disclosure may be conventional or full potential milk replacers. Disclosed conventional milk replacers may include at least about 20 percent protein by dry weight. Disclosed full potential milk replacers may include about 25 to about 31 percent protein by dry weight. According to the present disclosure, the protein of either a conventional or a full potential milk replacer is provided by a combination of non-milk proteins (including textured soy proteins, described above) and milk-derived proteins.

Some or all of the non-milk protein source may be textured soy protein. Non-milk proteins, some or all of which is textured soy protein, may provide about 5 percent to about 70 percent of the protein by dry weight, about 5 percent to about 60 percent, about 5 percent to about 50 percent, about 5 percent to about 40 percent, about 5 percent to about 30 percent, about 5 percent to about 20 percent, about 5 percent to about 10 percent, about 10 percent to about 70 percent, about 20 percent to about 70 percent, about 30 percent to about 70 percent, about 40 percent to about 70 percent, about 50 percent to about 70 percent, about 60 percent to about 70 percent, or about 40 percent to about 50 percent of the protein by dry weight.

The non-milk proteins, some or all of which is textured soy protein, are present at least about 4 percent or at least about 5 percent (also described as “percentage units”) of the milk replacer. In some embodiments, the non-milk proteins, some or all of which is textured soy protein, are present at about 4 percent to about 40 percent, about 4 percent to about 35 percent, about 4 percent to about 30 percent, about 4 percent to about 25 percent, about 4 percent to about 20 percent, about 4 percent to about 15 percent, about 4 percent to about 10 percent, about 10 percent to about 40 percent, about 15 percent to about 40 percent, about 20 percent to about 40 percent, about 25 percent to about 40 percent, about 30 percent to about 40 percent, about 10 percent to about 25 percent, or about 4 percent to about 15 percent by dry weight of the milk replacer.

Dairy-derived protein sources are generally referred to as milk proteins and may include whey; whey products such as whey protein concentrate and delactosed whey; casein; skim milk; sodium caseinate; and calcium caseinate.

Milk proteins may provide the balance of the protein content (other than from non-milk proteins) of a milk replacer. For example, about 30 percent to about 95 percent of the protein by dry weight may be provided by at least one milk protein.

Fat in the milk replacers of the present disclosure may be provided at levels from about 10 to about 25 percent by dry weight of the milk replacer, such as about 20 percent fat by dry weight. Fat may be added as a dry fat powder where protein is used to encapsulate the fat droplet. Fat used in milk replacers may be of animal or of vegetable origin.

Methods of Manufacturing Textured Soy Proteins for Milk Replacers

Textured soy proteins for milk replacers may be produced from a soy starting material, such as soy flour, soy meal, soy white flakes, or soy protein concentrate. The soy flour may be defatted soy flour, which may be produced from soy white flakes, which may or may not have been heat treated. The soy meal may also be produced from heat-treated soy white flakes.

The soy starting material may be extruded such that at least a portion of the proteins therein are at least partially denatured, as described above. After extrusion, the textured soy protein may be dried to <20 percent moisture, <15 percent moisture, <12 percent moisture, <10 percent moisture, or <8 percent moisture.

After extrusion and drying, the textured soy protein may be ground. Grinding may also further dry the textured soy protein. For example, the textured soy protein may be dried during grinding to <10 percent moisture, <8 percent moisture, <6 percent moisture, or <4 percent moisture. In one example, grinding is performed by an air classifying grinder, which may utilize an air classifier to recirculate particles that are larger than desired back into the grinding zone or may maintain the particles that are larger than desired in the grinding zone while vacuuming off the smaller particles.

The textured soy protein produced by the methods disclosed herein may fit the definition of AAFCO 84.64 or the Handbook of Food Proteins, as described above.

The textured soy protein may form part of a dry milk replacer powder. Such powder is hydrated in water before being offered to young animals.

Methods of Feeding Milk Replacers Containing Textured Soy Proteins

Milk replacers disclosed herein include at least about 20 percent protein by dry weight. The textured soy protein is present at about 4 percent to about 40 percent by dry weight of the milk replacer. The textured soy protein may have been extruded, may have been ground, and/or may be substantially free of anti-nutritional factors.

Milk replacers containing textured soy protein and, optionally, other non-milk proteins, may be fed at a rate of about 1 pound per head per day to about 1.8 pounds per head per day. In a full potential setting, the milk replacer may be fed at a rate of at least about 1.8 pounds per head per day to about 3 pounds per head per day. Generally, animals are offered a fixed amount of milk replacer per day, which may form all or a portion of the young animal's daily feed ration. In addition, the milk replacer in the feed ration may be offered twice per day, and may generally be divided into equal parts. The amount of milk replacer that is offered may change over time as the young animal grows.

In addition to milk replacer, starter feed may be offered to the young animals on an ad libitum basis. Starter feeds, such as calf starter feeds, may include a mixture of one or more of corn, soybean meal, wheat middlings, oats, molasses, fat, ground cotton seed hulls, distillers grains, calcium carbonate, salt, and macronutrients and micronutrients. The starter feed may include about 45 percent to about 50 percent coarse ingredients such as corn, soy, and oats; about 16 percent to about 22 percent protein; about 2 percent to about 3 percent fat; about 5 percent to about 6 percent fiber (determined on an NIR basis); about 7 percent acid detergent fiber; about 6 percent molasses; and the balance including a mixture of other nutrients.

Methods of feeding milk replacers containing protein, of which about 5 percent to about 70 percent is composed of textured soy protein and, in some implementations, other non-milk proteins, may optimize performance through improved performance or at least through not negatively impacting animal performance in the first four weeks of life compared to young animals fed other soy protein sources in milk replacers containing the same level of protein at the same feeding rate.

Young animals are generally animals that are not weaned and may include calves, lambs, kids, other ruminants, piglets, other young livestock animals, foals, young zoo animals, and young companion animals. Typically, young animals ingest milk replacer during the first six to eight weeks of life and sometimes up to twelve weeks of life. In some implementations of the present disclosure, the young animal ingests the milk replacer containing textured soy protein for at least four consecutive weeks beginning at a young age. The young animal may begin ingesting the milk replacer at or close to birth such as within a few days from birth, e.g., 1-5 days from birth. Thus, young animals may be offered milk replacer from about birth (e.g., about 1-5 days from birth) for at least twenty-eight consecutive days according to methods of the present disclosure.

With respect to improved performance, young animals ingesting the milk replacers that include textured soy protein, for at least twenty-eight days from about birth, have been shown to have equal or improved performance compared to young animals fed milk replacers with the same amount of total protein and same amount of soy protein, but the soy protein is from other than textured soy protein. This improved performance is shown generally through improved growth of the young animal along with no reduction in calf health. Such improved performance may involve increased total weight gain and/or an improved feed-to-gain ratio. Additionally or alternatively, improved performance may be observed through any one or more of increased growth (as indicated by, for example, hip height, heart girth, body length, and body volume), and better health (as indicated by, for example, scour scores and scour days).

### EXAMPLES

The following Examples illustrate various aspects of the disclosed compositions and in methods and are in no way limiting of such compositions or methods. Examples 1-4 below are illustrative of the effects of feeding young animals milk replacers that include soy-derived proteins.

Example 1: This example demonstrates the improved performance of calves following consumption of a milk replacer that included textured soy protein compared to a milk replacer that included hydrolyzed soy protein modified.

Materials and Methods: Forty two three- to ten-day-old Holstein bull calves from Wisconsin, weighing approximately 90 to 110 pounds each, were assigned by weight and initial gamma globulin level to one of two treatment groups. The gamma globulin ranges were <0.49 percent, 0.50 percent to 0.99 percent, 1.00 percent to 1.49 percent, 1.50 percent to 2.49 percent, and >2.5 percent gamma globulin by weight of serum.

Twenty four calves were assigned to a control group (“HSPM”) fed milk replacer having 20 weight percent fat and 20 weight percent protein, 50 percent of which (i.e., 10 percentage units by dry weight of the milk replacer) was from hydrolyzed soy protein modified (Glymaxene®, Land O'Lakes, Arden Hills, Minn.).

Eighteen calves were assigned to a test group (“TSP”) fed milk replacer having 20 weight percent fat and 20 weight percent protein, 50 percent of which (i.e., 10 percentage units by dry weight of the milk replacer) was from a commercially available textured soy protein (Cargill, Minneapolis, Minn.). The textured soy protein had been ground to ≤44 μm and had a protein content of about 51 percent.

Calves in both groups were fed 0.75 lb milk replacer (control HSPM or test TSP formulation, as applicable) per feeding during days 1-42. The milk replacer was fed twice a day through day 35, then once a day through day 42.

Calves were weighed upon arrival and weekly thereafter. Other performance parameters were measured or calculated daily and summarized weekly as well as at the end of the trial. Performance data were analyzed by GLM. Data were reported as LSMEANS and separated by the PDIFF function of SAS.

Results: The results of Example 1 are provided in Table 1 below.

TABLE 1

| | | | |
|---|---|---|---|
| Treatment: | HSPM | TSP | SE |

Calf Weights (lb)

| | | | |
|---|---|---|---|
| Initial Weight | 102.0 | 100.9 | 1.76 |
| Week 3 Weight | 113.4 | 113.9 | 2.42 |
| Week 6 Weight | 148.0 | 148.7 | 4.58 |

Gain (lb)

| | | | |
|---|---|---|---|
| Week 3 | 7.96 | 7.89 | 0.69 |
| Week 6 | 14.68 | 13.42 | 1.28 |
| Total Gain (lb) | 45.92 | 47.79 | 3.98 |

Milk Replacer Consumption (DM Basis) (lb)

| | | | |
|---|---|---|---|
| Week 3 | 10.43 | 10.45 | 0.05 |
| Week 6 | 5.25 | 5.25 | |
| Total Milk Replacer Consumption (lb) | 56.60 | 56.63 | 0.37 |

Starter Consumption (DM Basis) (lb)

| | | | |
|---|---|---|---|
| Week 3 | 3.76 | 3.13 | 0.65 |
| Week 6 | 18.22 | 19.25 | 1.70 |
| Total Dry Feed Consumption (lb) | 39.52 | 39.51 | 4.52 |
| Average Feed:Gain | 2.17 | 2.06 | 0.11 |

Summary: Total gain after 6 weeks was 45.90 lb for calves fed the control milk replacer having hydrolyzed soy protein modified and 47.79 lb for the test milk replacer having textured soy protein. Calves fed the milk replacer having textured soy protein demonstrated an improved feed-to-gain ratio (2.06) compared to the control milk replacer (2.17).

Conclusion: This example again demonstrates the improved performance of calves fed a milk replacer that included textured soy protein compared to a milk replacer that included hydrolyzed soy protein modified. Improved performance included increased total gain and improved feed-to-gain ratio.

Example 2: This second trial again demonstrates the improved performance of calves following consumption of a milk replacer that included textured soy protein compared to a milk replacer that included hydrolyzed soy protein modified.

Materials and Methods: Seventy two three- to ten-day-old Holstein bull calves from Wisconsin, weighing approximately 80 to 105 pounds each, were assigned by weight and initial gamma globulin level to one of two treatment groups. The gamma globulin ranges were <0.49 percent, 0.50 percent to 0.99 percent, 1.00 percent to 1.49 percent, 1.50 percent to 2.49 percent, and >2.5 percent gamma globulin by weight of serum.

Thirty six calves were assigned to a control group (“HSPM”) fed milk replacer having 17 weight percent fat and 25 weight percent protein, 40 percent of which (i.e., 10 percentage units by dry weight of the milk replacer) was from hydrolyzed soy protein modified (Glymaxene®, Land O'Lakes, Arden Hills, Minn.).

Thirty six calves were assigned to a test group (“TSP”) fed milk replacer having 17 weight percent fat and 25 weight percent protein, 40 percent of which (i.e., 10 percentage units by dry weight of the milk replacer) was from a textured soy protein. The textured soy protein was manufactured by Land O'Lakes using soy bean meal (Hi-Pro Feeds, Fiona, Tex.) which had been ground to ≤149 μm, then extruded with enough pressure and shear to yield a plasticized product, which was then ground to ≤44 μm. The textured soy protein included a small amount of calcium carbonate and had a protein content of about 50 percent.

Calves in both groups were fed 0.75 lb milk replacer (control HSPM or test TSP formulation, as applicable) per feeding during days 1-42. The milk replacer was fed twice a day through day 35, then once a day through day 42.

Calves were weighed upon arrival and weekly thereafter. Other performance parameters were measured or calculated daily and summarized weekly as well as at the end of the trial. Performance data were analyzed by GLM. Data were reported as LSMEANS and separated by the PDIFF function of SAS.

Results: The results of Example 2 are provided in Table 2 below.

TABLE 2

| Treatment: | HSPM | TSP | SE |
|---|---|---|---|
| Initial Ig | 3.85 | 3.58 | 0.23 |

Calf Weights (lb)

| | | | |
|---|---|---|---|
| Initial Weight | 101.4 | 102.8 | 1.06 |
| Week 3 Weight | 115.7 | 118.8 | 1.37 |
| Week 6 Weight | 142.9 | 147.1 | 2.63 |

Gain (lb)

| | | | |
|---|---|---|---|
| Week 3 | 5.84 | 6.41 | 0.61 |
| Week 6 | 9.73 | 11.36 | 1.15 |
| Total Gain (lb) | 41.44 | 44.39 | 2.29 |

Milk Replacer Consumption (DM Basis) (lb)

| | | | |
|---|---|---|---|
| Week 3 | 10.43 | 10.37 | 0.06 |
| Week 6 | 5.25 | 5.25 | |
| Total Milk Replacer Consumption (lb) | 55.74 | 55.81 | 0.36 |

Starter Consumption (DM Basis) (lb)

| | | | |
|---|---|---|---|
| Week 3 | 1.21 | 1.72 | 0.28 |
| Week 6 | 11.87 | 12.09 | 1.21 |
| Total Dry Feed Consumption (lb) | 21.02 | 22.21 | 2.64 |
| Average Feed:Gain | 1.89 | 1.81 | 0.06 |

Summary: Total gain after 6 weeks was 41.44 lb for calves fed the control milk replacer having hydrolyzed soy protein modified and 44.39 lb for the test milk replacer having textured soy protein. Calves fed the milk replacer having textured soy protein demonstrated an improved feed-to-gain ratio (1.81) compared to the control milk replacer (1.89).

Conclusion: Examples 1 and 2 demonstrate the improved performance of calves fed a milk replacer that included textured soy protein compared to a milk replacer that included hydrolyzed soy protein modified. Improved performance included increased total gain and improved feed-to-gain ratio.

Example 3: This third trial further demonstrates the improved performance of calves following consumption of a milk replacer that included textured soy protein compared to a milk replacer that included hydrolyzed soy protein modified.

Materials and Methods: One hundred and two three- to ten-day-old Holstein bull calves from Wisconsin, weighing approximately 80 to 105 pounds each, were assigned by weight and initial gamma globulin level to one of two treatment groups. The gamma globulin ranges were <0.49 percent, 0.50 percent to 0.99 percent, 1.00 percent to 1.49 percent, 1.50 percent to 2.49 percent, and >2.5 percent gamma globulin by weight of serum.

Fifty one calves were assigned to a control group (“HSPM”) fed milk replacer having 17 weight percent fat and 25 weight percent protein, 40 percent of which (i.e., 10 percentage units by dry weight of the milk replacer) was from hydrolyzed soy protein modified (Glymaxene®, Land O'Lakes, Arden Hills, Minn.).

Fifty one calves were assigned to a test group (“TSP”) fed milk replacer having 17 weight percent fat and 25 weight percent protein, 40 percent of which (i.e., 10 percentage units by dry weight of the milk replacer) was from a commercially available textured soy protein (Cargill, Minneapolis, Minn.). The textured soy protein had been ground to ≤44 μm and had a protein content of 25 percent. Compared to the textured soy protein of Example 2, the textured soy protein of Example 3 did not include added calcium carbonate, and did include a low amount (i.e., about 0.01%) of citric acid.

Calves in both groups were fed 0.75 lb milk replacer (control or test formulation, as applicable) per feeding during days 1-42. The milk replacer was fed twice a day through day 35, then once a day through day 42.

Calves were weighed upon arrival and weekly thereafter. Other performance parameters were measured or calculated daily and summarized weekly as well as at the end of the trial. Performance data were analyzed by GLM. Data were reported as LSMEANS and separated by the PDIFF function of SAS.

Results: The results of Example 3 are provided in Table 3 below.

TABLE 3

| Treatment: | HSPM | TSP | SE |
|---|---|---|---|
| Initial Ig | 3.44 | 3.50 | 0.17 |

Calf Weights (lb)

| | | | |
|---|---|---|---|
| Initial Weight | 95.7 | 99.5 | 0.83 |
| Week 3 Weight | 108.0 | 108.7 | 1.04 |
| Week 6 Weight | 140.7 | 141.2 | 1.98 |

Gain (lb)

| | | | |
|---|---|---|---|
| Week 3 | 7.19 | 7.29 | 0.46 |
| Week 6 | 11.48 | 11.92 | 0.74 |
| Total Gain (lb) | 44.96 | 45.78 | 1.83 |

Milk Replacer Consumption (DM Basis) (lb)

| | | | |
|---|---|---|---|
| Week 3 | 10.44 | 10.43 | 0.03 |
| Week 6 | 5.25 | 5.25 | |
| Total Milk Replacer Consumption (lb) | 54.10 | 54.06 | 0.37 |

Starter Consumption (DM Basis) (lb)

| | | | |
|---|---|---|---|
| Week 3 | 1.21 | 1.13 | 0.19 |
| Week 6 | 15.33 | 14.61 | 0.94 |
| Total Dry Feed Consumption (lb) | 27.31 | 25.58 | 2.05 |
| Average Feed:Gain | 1.90 | 1.79* | 0.06 |

Summary: Total gain after 6 weeks was 44.96 lb for calves fed the control milk replacer having hydrolyzed soy protein modified and 45.78 lb for the test milk replacer having textured soy protein. Calves fed the milk replacer having textured soy protein demonstrated an improved feed-to-gain ratio (1.79) compared to the control milk replacer (1.90) (*P<0.20). Neither the exclusion of calcium carbonate nor the addition of acid to the textured soy protein had a significant effect on the results.

Conclusion: This example further demonstrates the improved performance of calves fed a milk replacer that included textured soy protein compared to a milk replacer that included hydrolyzed soy protein modified. Improved performance included increased total gain and improved feed-to-gain ratio.

Example 4: In another trial, calves were fed milk replacer including one of defatted soy flour, hydrolyzed soy protein modified, or textured soy protein.

Materials and Methods: One hundred and fifty three- to ten-day-old Holstein bull calves from Wisconsin, weighing approximately 80 to 105 pounds each, were assigned by weight and initial gamma globulin level to one of three treatment groups. The gamma globulin ranges were <0.49 percent, 0.50 percent to 0.99 percent, 1.00 percent to 1.49 percent, 1.50 percent to 2.49 percent, and >2.5 percent by weight gamma globulin by weight of serum.

Fifty calves were assigned to a negative control group (“DSF”) fed milk replacer having 17 weight percent fat and 25 weight percent protein, 60 percent of which was milk protein and 40 percent of which (i.e., 10 percentage units by dry weight of the milk replacer) was from defatted soy flour. The defatted soy protein had a protein content of 50.4 percent, a Protein Dispersibility Index (PDI) of 20, and had been heat treated, which can reduce the amount of active anti-nutritional factors compared to defatted soy flour that has not been heat treated.

Fifty calves were assigned to a negative control group (“HSPM”) fed milk replacer having 17 weight percent fat and 25 weight percent protein, 60 percent of which was milk protein and 40 percent of which (i.e., 10 percentage units by dry weight of the milk replacer) was from hydrolyzed soy protein modified (Glymaxene®, Land O'Lakes, Arden Hills, Minn.). The hydrolyzed soy protein modified had a protein content of 50.5 percent.

Fifty calves were assigned to a test group (“TSP”) fed milk replacer having 17 weight percent fat and 25 weight percent protein, 60 percent of which was milk protein and 40 percent of which (i.e., 10 percentage units by dry weight of the milk replacer) was from a commercially available textured soy protein (Cargill, Minneapolis, Minn.). The textured soy protein had been ground to ≤44 μm and had a protein content of 50.9 percent.

On Days 1-7, calves in all groups were fed twice daily at a rate of 0.95 lb of the applicable milk replacer (reconstituted to about 13 percent solids) per feeding. The milk replacer was fed twice a day through day 35, then once a day through day 42. On Days 8-28, calves were fed twice daily at a rate of 1.25 lb of the applicable milk replacer (again reconstituted to about 13 percent solids) per feeding. Warm water was offered between feedings through Day 14.

Calves were weighed upon arrival and at each of Weeks 2-4. Initial and final hip height, heart girth, and body length were recorded, and body volume calculated. The quantity of milk replacer consumed or refused by each calf was recorded daily throughout the study. These values were converted to a dry matter (DM) basis.

Results: The results of Example 4 are provided in Table 4 below.

TABLE 4

| Treatment: | DSF | HSPM | TSP | P if <0.20 | SE |
|---|---|---|---|---|---|
| Initial Ig | 3.84 | 3.77 | 3.93 | | 0.19 |

Calf Weights (lb)

| | | | | | |
|---|---|---|---|---|---|
| Initial Weight | 103.8 | 102.9 | 102.4 | | 1.25 |
| Week 2 Weight | 103.9a | 115.4b | 115.0b | 0.01 | 1.47 |
| Week 3 Weight | 110.9a | 127.5b | 127.4b | 0.01 | 1.58 |
| Week 4 Weight | 116.4a | 137.2b | 137.7b | 0.01 | 1.79 |
| Gain (lb) | | | | | |
| Weeks 1 & 2 | 0.04a | 12.47b | 12.56b | 0.01 | 0.82 |
| Week 3 | 7.07a | 12.13b | 12.36b | 0.01 | 0.50 |
| Week 4 | 5.44a | 9.67b | 10.30b | 0.01 | 0.48 |
| Total Gain (lb) | 12.55a | 34.27b | 35.22b | 0.01 | 1.30 |

Milk Replacer Consumption (DM Basis) (lb)

| | | | | | |
|---|---|---|---|---|---|
| Weeks 1 & 2 | 25.86a | 27.98b | 26.80ab | 0.02 | 0.53 |
| Week 3 | 16.45a | 17.35b | 17.24b | 0.01 | 0.18 |
| Week 4 | 16.69a | 17.44b | 17.47b | 0.01 | 0.14 |
| Total Milk Replacer Consumption (lb) | 59.00a | 62.77b | 61.51b | 0.01 | 0.69 |
| Average Feed to Gain | 2.59b | 1.88a | 1.76a | <0.01 | 0.05 |
| Feed to Gain, HSPM vs. TSP | | | | 0.08 | 0.05 |

Hip Height (cm)

| | | | | | |
|---|---|---|---|---|---|
| Initial Hip Height | 84.8 | 84.9 | 84.2 | | 0.43 |
| Day 28 | 87.3a | 89.4b | 89.2b | 0.01 | 0.42 |
| Hip Height Gain (cm) | 2.51a | 4.47b | 5.12b | 0.01 | 0.36 |

Heart Girth (cm)

| | | | | | |
|---|---|---|---|---|---|
| Initial Heart Girth | 79.66 | 79.32 | 79.70 | | 0.35 |
| Day 28 | 84.2a | 88.8b | 88.8b | 0.01 | 0.52 |
| Hearth Girth Gain (cm) | 4.58a | 9.49b | 9.02b | 0.01 | 0.43 |

Body Length (cm)

| | | | | | |
|---|---|---|---|---|---|
| Initial Body Length | 78.41 | 78.69 | 77.68 | | 0.53 |
| Day 28 | 83.5a | 87.6b | 86.9b | 0.01 | 0.57 |
| Body Length Gain (cm) | 5.11a | 8.95b | 9.31b | 0.01 | 0.69 |

Body Volume (L)

| | | | | | |
|---|---|---|---|---|---|
| Initial Body Volume | 168.9 | 168.8 | 166.1 | | 2.08 |
| Day 28 | 196.3a | 221.7b | 219.7b | 0.01 | 2.95 |
| Body Volume Gain (L) | 27.33a | 52.82b | 53.52b | 0.01 | 2.49 |

Scour Score

| | | | | | |
|---|---|---|---|---|---|
| Average 2 Week | 1.80 | 1.79 | 1.76 | | 0.04 |
| Week 3 | 1.34b | 1.07a | 1.08a | 0.01 | 0.04 |
| Week 4 | 1.21b | 1.01a | 1.00a | 0.01 | 0.03 |
| Average Scour Score | 1.54b | 1.41a | 1.40a | 0.01 | 0.03 |

Scour Days

| | | | | | |
|---|---|---|---|---|---|
| Total 2 Week | 10.13 | 10.21 | 9.69 | | 0.42 |
| Week 3 | 2.32b | 0.51a | 0.55a | 0.01 | 0.27 |
| Week 4 | 1.42b | 0.05a | 0.00a | 0.01 | 0.21 |
| Total Scour Days | 13.87b | 10.77a | 10.24a | 0.01 | 0.65 |

Means in the same row not followed by a common letter differ (P < .05) using LSD procedure.

Summary: Calves fed the test milk replacer with textured soy protein performed better in all measurements than calves fed the negative control milk replacer with defatted soy flour. Total gain after 4 weeks was 12.55 lb for calves fed the negative control milk replacer with defatted soy flour, 34.27 lb for the control milk replacer with hydrolyzed soy protein modified (P=0.01), and 35.22 lb for the test milk replacer with textured soy protein. Calves fed the milk replacer with textured soy protein demonstrated an improved feed-to-gain ratio (1.76) compared to the defatted soy flour milk replacer (2.59) (P<0.01) and compared to the hydrolyzed soy protein modified milk replacer (1.88) (P=0.08).

Calves fed the test milk replacer with textured soy protein demonstrated increased growth, including total hip height gain, total heart girth gain, total body length gain, and total body volume gain, compared to calves fed the negative control milk replacer with defatted soy flour (P=0.01 for all). Calves fed the test milk replacer also demonstrated increased health, including a lower average scour score and fewer total scour days compared to calves fed the negative control milk replacer with defatted soy flour (P=0.01 for both).

Conclusion: This example demonstrates the improved performance of calves fed a milk replacer that included textured soy protein compared to a milk replacer that included defatted soy flour or hydrolyzed soy protein modified. The improved response was statistically significant compared to defatted soy flour and at least numerically greater than hydrolyzed soy protein modified. Improved performance included increased total gain, improved feed-to-gain ratio, increased growth (as shown by hip height, heart girth, body length, and body volume), and better health (as shown by scour scores and scour days). Without being limited to any mechanism or mode of action, the improved performance may be a result of reducing or eliminating anti-nutritional factors in textured soy protein compared to defatted soy flour.

As used herein, the term “about” modifying, for example, the quantity of a component in a composition, concentration, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

Similarly, it should be appreciated that in the foregoing description of example embodiments, various features are sometimes grouped together in a single embodiment for the purpose of streamlining the disclosure and aiding in the understanding of one or more of the various aspects. These methods of disclosure, however, are not to be interpreted as reflecting an intention that the claims require more features than are expressly recited in each claim. Rather, as the following claims reflect, inventive aspects lie in less than all features of a single foregoing disclosed embodiment, and each embodiment described herein may contain more than one inventive feature.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them, and variations, modifications, additions, and improvements are possible. More generally, embodiments in accordance with the present disclosure have been described in the context or particular embodiments. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A milk replacer for young animals, the milk replacer comprising at least about 20 percent protein by dry weight, wherein at least about 5 percentage units of the protein by dry weight is provided by a ground, extruded textured soy protein substantially free of anti-nutritional factors.

2. The milk replacer of claim 1, wherein at least about 65 percent of the textured soy protein passes through a 37 μm mesh.

3. The milk replacer of claim 1, wherein about 5 percent to about 70 percent of the protein by dry weight is provided by the textured soy protein.

4. The milk replacer of claim 1, wherein about 30 percent to about 95 percent of the protein by dry weight is provided by at least one milk protein.

5. The milk replacer of claim 1, wherein the textured soy protein comprises about 20 percent to about 60 percent by weight protein.

6. The milk replacer of claim 1, wherein the textured soy protein is present at about 5 percent to about 40 percent by dry weight of the milk replacer.

7. The milk replacer of claim 1, wherein at least a portion of protein of the textured soy protein is at least partially denatured.

8. The milk replacer of claim 1, wherein the textured soy protein is suspended in the milk replacer when the milk replacer is hydrated.

9. A method of feeding a young animal comprising:
providing a milk replacer to the young animal, the milk replacer comprising at least about 20 percent protein by dry weight, wherein at least about 5 percentage units of the protein by dry weight is provided by a ground, extruded textured soy protein substantially free of anti-nutritional factors.

10. The method of claim 9, wherein at least about 65 percent of the textured soy protein passes through a 37 μm mesh.

11. The method of claim 9, wherein the young animal experiences improved performance in response to ingesting the milk replacer.

12. The method of claim 11, wherein improved performance comprises an increased feed-to-gain ratio.

13. The method of claim 11, wherein performance improves by six weeks after birth or earlier.

14. The method of claim 9, wherein the young animal is a calf.

15. A method of feeding a young animal comprising:
providing a textured soy protein in a milk replacer, the textured soy protein present at about 4 percent to about 40 percent by dry weight of the milk replacer and the textured soy protein having been extruded and ground; and
providing the milk replacer to the young animal.

16. The method of claim 15, wherein at least about 65 percent of the textured soy protein passes through a 37 μm mesh.

17. The method of claim 15, wherein the textured soy protein is suspended in the milk replacer when the milk replacer is hydrated.

18. The method of claim 15, wherein the textured soy protein comprises about 20 percent to about 60 percent by weight protein.

19. The method of claim 15, wherein the milk replacer powder comprises about 20 percent to about 30 percent protein by dry weight.

20. The method of claim 15, wherein the young animal experiences improved performance in response to ingesting the milk replacer.

21. The method of claim 20, wherein the improved performance comprises an increased feed-to-gain ratio.
